SEQUENCE LISTING (6 sequences)

SEQ ID NO: 1 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 2 - DNA, 1377 bp, Stevia rebaudiana
atggaaaacaagaccgaaacaacagttagacgtaggcgtagaatcattctgtttccagtaccttttcaagggcacatcaatccaatactacaactagccaacgttttgtactctaaaggtttttctattacaatctttcacaccaatttcaacaaaccaaaaacatccaattacccacatttcacattcagattcatacttgataatgatccacaagatgaacgtatttcaaacttacctacccacggtcctttagctggaatgagaatcccaatcatcaatgaacatggtgccgatgagcttagaagagaattagagttacttatgttggcatccgaagaggacgaggaagtctcttgtctgattactgacgctctatggtactttgcccaatctgtggctgatagtttgaatttgaggagattggtactaatgacatccagtctgtttaactttcacgctcatgttagtttaccacaatttgacgaattgggatacttggaccctgatgacaagactaggttagaggaacaggcctctggttttcctatgttgaaagtcaaagatatcaagtctgcctattctaattggcaaatcttgaaagagatcttaggaaagatgatcaaacagacaaaggcttcatctggagtgatttggaacagtttcaaagagttagaagagtctgaattggagactgtaatcagagaaattccagcaccttcattcctgataccattaccaaaacatttgactgcttcctcttcctctttgttggatcatgacagaacagtttttcaatggttggaccaacaaccacctagttctgttttgtacgtgtcatttggtagtacttctgaagtcgatgaaaaggacttccttgaaatcgcaagaggcttagtcgatagtaagcagtcattcctttgggtcgtgcgtccaggtttcgtgaaaggctcaacatgggtcgaaccacttccagatggttttctaggcgaaagaggtagaatagtcaaatgggttcctcaacaggaagttttagctcatggcgctattggggcattctggactcattccggatggaactcaactttagaatcagtatgcgaaggggtacctatgatcttttcagattttggtcttgatcaaccactgaacgcaagatacatgtctgatgttttgaaagtgggtgtatatcttgaaaatggctgggaaaggggtgaaatagctaatgcaataagacgtgttatggttgatgaagagggggagtatatcagacaaaacgcaagagtgctgaagcaaaaggccgacgtttctctaatgaagggaggctcttcatacgaatccttagaatctcttgtttcctacatttcatcactgtaa

SEQ ID NO: 3 - protein, 462 aa, Oryza sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 4 - DNA, 1389 bp, Oryza sativa
atggatagtggctactcctcatcttatgctgctgccgctggtatgcacgttgtgatctgcccttggttggcctttggtcacctgttaccatgtctggatttagcccaaagactggcctcaagaggccatagagtatcatttgtgtctactcctagaaatatctctcgtttaccaccagtcagacctgctctagctcctctagttgcattcgttgctcttccacttccaagagtagaaggattgccagacggcgctgaatctactaatgacgtaccacatgatagacctgacatggtcgaattgcatagaagagcctttgatggattggcagctccattttctgagttcctgggcacagcatgtgcagactgggttatagtcgatgtatttcatcactgggctgctgcagccgcattggaacataaggtgccttgtgctatgatgttgttagggtcagcacacatgatcgcatccatagctgatagaagattggaaagagctgaaacagaatccccagccgcagcaggacaaggtaggccagctgccgccccaacctttgaagtggctagaatgaaattgattcgtactaaaggtagttcagggatgagtcttgctgaaaggttttctctgacattatcaagatcatcattagttgtaggtagatcctgcgtcgagttcgaacctgaaacagtacctttactatctactttgagaggcaaacctattactttccttggtctaatgcctccattacatgaaggaaggagagaagatggtgaagatgctactgttaggtggttagatgcccaacctgctaagtctgttgtttacgttgcattgggttctgaggtaccactaggggtggaaaaggtgcatgaattagcattaggacttgagctggccggaacaagattcctttgggctttgagaaaaccaaccggtgtttctgacgccgacttgctaccagctgggttcgaagagagaacaagaggccgtggtgtcgttgctactagatgggtcccacaaatgagtattctagctcatgcagctgtaggggcctttctaacccattgcggttggaactcaacaatagaaggactgatgtttggtcatccacttattatgttaccaatctttggcgatcagggacctaacgcaagattgattgaggcaaagaacgcaggtctgcaggttgcacgtaatgatggtgatggttcctttgatagagaaggcgttgcagctgccatcagagcagtcgccgttgaggaagagtcatctaaagttttccaagctaaggccaaaaaattacaagagattgtggctgacatggcttgtcacgaaagatacatcgatggtttcatccaacaattgagaagttataaagactaa

SEQ ID NO: 5 - protein, 808 aa, Arabidopsis thaliana
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 6 - DNA, 2427 bp, Arabidopsis thaliana
atggcgaacgcggagcgtatgatcacccgtgttcacagccagcgcgaacgtctgaatgaaactctggtttctgaacgcaacgaagtactggctttgctgagccgcgttgaagcgaagggcaaaggtatcctccagcagaaccaaattatcgcggaatttgaggcactgccggaacagacccgcaaaaaactggaaggtggcccgttcttcgacttgctgaaatctacccaggaagcaattgttctgccgccgtgggttgctctggccgttcgtccgcgcccgggtgtgtgggaatacctgcgtgttaacctgcacgcgctggtcgtcgaagaactgcaaccggctgaattcctgcatttcaaagaagaactggtggatggtgtcaaaaacggcaactttaccctggagctggactttgaaccgttcaacgcatcgatcccgcgtccgacactgcacaaatacatcgggaacggcgtcgacttcctgaaccgtcacctgagcgctaagctgttccacgacaaagaatcactgctgccactgctgaaatttctgcgcctgcactcccaccagggtaaaaacctgatgctgtccgaaaaaatccagaacctgaacacactgcagcacaccctgcgtaaagcggaagaatacctggctgagctgaaaagcgaaacgctgtacgaagaattcgaagccaaattcgaagaaatcggcctcgaacgtggctggggcgacaacgctgaacgtgttctcgacatgattcgcctgctgctggacctgctggaagctccggacccatgcactcttgaaaccttcctgggccgtgtaccgatggtgttcaacgttgttattctgtctccgcacggttacttcgcacaggacaacgttctgggctatcctgacacgggcggccaggttgtctacattctggatcaggtacgtgctctggagatcgagatgctgcagcgtatcaaacagcagggtttgaacatcaagccgcgtatcctcatcttgacacgtctgctcccggatgcggtcgggaccacttgcggtgagcgtctggaacgcgtttacgattctgaatactgcgatatcctgcgtgtaccgttccgtacggaaaaaggtattgtgcgtaaatggatctctcgcttcgaggtttggccgtatctggagacgtacaccgaagatgctgctgtggaactgagcaaagaactgaacggtaaaccggatctgatcattggtaactatagcgacggcaatctggtagcttcgctgctggctcacaaactgggtgttacgcagtgtaccatcgcacacgcactggaaaaaaccaagtacccggactctgacatctactggaaaaaactggacgataaatatcatttctcttgtcagttcaccgccgacatcttcgccatgaaccatacggacttcatcattacctcgaccttccaggaaatcgccggttctaaagaaaccgtgggccagtatgaaagtcacactgcgtttactttaccggggctgtatcgcgttgtgcacggtatcgatgttttcgacccgaaatttaacattgtttctccgggtgcagatatgtcaatctatttcccgtacaccgaggaaaaacgtcgcctgactaaattccactctgaaattgaagaactgctgtactccgacgtggagaacaaggaacacctgtgtgtactgaaggacaagaaaaaaccgatcctgttcacgatggcgcgtctggaccgtgtaaaaaaccttagcggccttgtagaatggtatggcaagaacacccgcctgcgtgagcttgccaacttagtggtggttggcggtgaccgtcgtaaagaatctaaagacaacgaagaaaaagccgaaatgaaaaaaatgtacgacctcatcgaggaatacaagctgaacggtcagtttcgttggatctcctcccagatggatcgtgtacgtaacggtgaactgtatcgttacatttgcgatactaaaggcgctttcgttcagccggcactgtacgaagccttcggtctgaccgtagttgaagcgatgacttgcggtctgccgactttcgcgacttgcaaaggcggcccggctgaaatcatcgtacatggcaagtcgggtttccatatcgatccgtaccacggtgaccaggcggctgacaccctcgctgatttcttcacgaaatgtaaagaagacccaagccactgggatgaaatctccaaagggggcttacagcgtatcgaagaaaaatacacctggcagatctactcccagcgcctgctgactctgaccggggtttacggtttttggaaacacgtcagcaacctggatcgtctggaagcccgtcgttacctggaaatgttttatgcgctgaaatatcgtccactcgctcaggcagttccgctggcgcaggacgattaa

(19) United States
(12) Patent Application Publication
(10) Pub. No.: US 2018/0320211 A1
(43) Pub. Date: Nov. 8, 2018

(54) METHOD FOR PREPARING REBAUDIOSIDE M BY USING SACCHAROMYCES CEREVISIAE ENZYMATIC PROCESS

(71) Applicant: PepsiCo, Inc., Purchase, NY (US)

(72) Inventors: Haomian DU, Jiangsu (CN); Xihuan WEI, Jiangsu (CN); Xinkai XIE, Jiangsu (CN); Junhua TAO, Jiangsu (CN)

(21) Appl. No.: 15/740,572

(22) PCT Filed: Aug. 21, 2015
(86) PCT No.: PCT/CN2015/087751

§ 371 Date: Jul. 30, 2018

(30) Foreign Application Priority Data
Jun. 30, 2015 (CN) 201510375211.8

(51) Int. Cl. C12P 19/56

(52) U.S. Cl. CPC C12P 19/56; C12Y 204/01017

(57) ABSTRACT

Provided in the present invention is a method for preparing rebaudioside M by using an enzymatic process, which catalyzes the formation of rebaudioside M using a recombinant Saccharomyces cerevisiae containing a UDP-glucosyltransferase or a UDP-glucosyltransferase prepared therefrom. The recombinant Saccharomyces cerevisiae is obtained by transforming an expression vector containing the UDP-glucosyltransferase gene under the control of a strong promoter.

### FIELD OF INVENTION

The present invention belongs to the field of bioengineering enzymes, and in particular, to a method for preparing rebaudioside M by using a Saccharomyces cerevisiae process.

### BACKGROUND

Sweetening agent, a type of food additives widely applied to food, beverages, and candies production, may be added during the food production process, and may also be appropriately diluted to replace sucrose during the home baking process. Sweetening agent comprises natural and artificial sweetening agents, with the former being, for example, sucrose, high fructose corn syrup, honey, or the like and the later being, for example, aspartame, saccharin, or the like.

Stevioside is a type of natural sweetening agent extracted from Stevia rebaudiana plant, which currently has been widely used in food and beverages. Extracts of Stevia rebaudiana contain a plurality of steviosides including rebaudioside; the different batch components of naturally extracted stevioside vary greatly, requiring subsequent purification. Current commercial products of rebaudioside A contain some other steviosides such as rebaudiosides C, D, and F or the like. Stevioside prepared by extraction method usually may have some impurities mixed therein, which may have certain influence on the range of its usage. Rebaudioside M has advantages compared with rebaudioside A, but it has a low content in Stevia rebaudiana leaves and is only detected in Stevia rebaudiana plant “Morita” (2010, J. Appl. Glycosci., 57, 199-209). Commercial production of rebaudioside M has not been made available at present.

Chinese patent CN103397064A discloses a method for enzymatically preparing rebaudioside M, in which in the presence of glucosyl donors, UDP-glucosyltransferase prepared by Escherichia coli or Escherichia coli containing UDP-glucosyltransferase is utilized to catalyze rebaudioside A or rebaudioside D to produce rebaudioside M. In this method, the genetic engineering bacteria, Escherichia coli, used for producing UDP-glucosyltransferase is not a safe strain (generally regarded as safe (GRAS)). This is because a toxin is produced during its cultivation process; thus the produced rebaudioside M therefrom cannot be used directly. Furthermore, while the Escherichia coli is prokaryote, the gene of UDP-glucosyltransferase originates from eukaryote.

Because of the complexity of the protein expression process of eukaryote and prokaryote, the expression of eukaryotic-derived enzymes by prokaryotic bacteria affects the activity of the enzyme. Saccharomyces cerevisiae is a known safe strain; but expression quantity of exogenous gene expression in Saccharomyces cerevisiae is low.

### SUMMARY

The present invention aims to solve the technical problem that the produced rebaudioside M in prior art has a safety concern which arises because a toxin is produced by engineering bacteria itself during the process for preparing rebaudioside M utilizing Escherichia coli containing UDP-glucosyltransferase. Provided in the present invention is a method for preparing rebaudioside M by utilizing a GRAS strain in an enzymatic process, and the method can produce a high purity rebaudioside M product at a lower cost and with a shorter cycle.

The following technical solution is employed by the present invention to solve the technical problem described above.

The present invention discloses a method for preparing rebaudioside M by utilizing Saccharomyces cerevisiae in an enzymatic process. In the presence of glucosyl donors, recombinant Saccharomyces cerevisiae containing UDP-glucosyltransferase or UDP-glucosyltransferase prepared therefrom is used to catalyze rebaudioside A or rebaudioside D for producing rebaudioside M; the recombinant Saccharomyces cerevisiae is constructed using the following method: introducing a strong promoter into a plasmid to obtain a vector plasmid; inserting the gene of the UDP-glucosyltransferase into the vector plasmid at an enzyme cutting site so as to obtain an expression vector under the control of the strong promoter; and then transforming the Saccharomyces cerevisiae to obtain the recombinant Saccharomyces cerevisiae.

In the foregoing method for preparing rebaudioside M, the UDP-glucosyltransferase is UGT-A from Stevia rebaudiana and/or UGT-B from Oryza sativa.

In the foregoing method for preparing rebaudioside M, an amino acid sequence of the UGT-A has at least 60% identity to an amino acid sequence shown by SEQ ID NO. 1 in a sequence listing; an amino acid sequence of the UGT-B from the Oryza sativa has at least 60% identity to an amino acid sequence shown by SEQ ID NO. 3 in the sequence listing.

In the foregoing method for preparing rebaudioside M, the amino acid sequence of the UGT-A is shown as SEQ ID NO. 1 in the sequence listing; the amino acid sequence of the UGT-B is shown as SEQ ID NO. 3 in the sequence listing.

In the foregoing method for preparing rebaudioside M, the enzyme cutting sites are HindIII and XbaI.

In the foregoing method for preparing rebaudioside M, the strong promoter is ADH2 or TEF1.

In the foregoing method for preparing rebaudioside M, the plasmid is pYES2.

In the foregoing method for preparing rebaudioside M, the method for constructing the vector plasmid is introducing an AgeI enzyme cutting site into the plasmid, and then introducing the strong promoter through the AgeI/HindIII site.

In the foregoing method for preparing rebaudioside M, the Saccharomyces cerevisiae is Saccharomyces cerevisiae BY4742.

In the foregoing method for preparing rebaudioside M, the glucosyl donors is UDP-glucose or UDP-glucose regeneration system consisted of sucrose, sucrose synthetase, and UDP.

In the foregoing method for preparing rebaudioside M, the recombinant Saccharomyces cerevisiae is affected by a cell-permeating agent to form recombinant Saccharomyces cerevisiae permeabilized cells for catalysis.

In the foregoing method for preparing rebaudioside M, the recombinant Saccharomyces cerevisiae is cultivated and then ultrasonically disrupted in an ice bath, and the crushed liquid is centrifuged and the supernatant is collected to obtain lyophilized powder of UGT-A or UGT-B for catalysis.

In the foregoing method for preparing rebaudioside M, the catalytic reaction is carried out in an aqueous system at a temperature of 4° C.˜50° C. and a pH of 5.0˜9.0.

Because of the implementation of the above technical solutions, the present invention has the following advantages compared with the prior art:
1. In the method for for preparing rebaudioside M by utilizing Saccharomyces cerevisiae in an enzymatic process, a catalytic production is carried out with UDP-glucosyltransferase produced from high safety recombinant Saccharomyces cerevisiae; the produced rebaudioside M is quite safe and may be used directly as food additives.
2. In the method for for preparing rebaudioside M by utilizing Saccharomyces cerevisiae in an enzymatic process, Saccharomyces cerevisiae is used as engineering bacteria; a strong promoter ADH2 or TEF1 is introduced into plasmid pYES2 through enzyme cutting site AgeI/HindIII to replace an original Gal promoter on the plasmid; an expression vector is constructed by inserting the UDP-glucosyltransferase gene from eukaryote into HindIII and XbaI. Because the gene of UDP-glucosyltransferase is under control of the strong promoter, it may be directly expressed without induction during the process for cultivating Saccharomyces cerevisiae BY4742, thus reducing fermentation time and the needed steps; and the activity of UDP-glucosyltransferase is higher than that produced from prokaryotic engineering bacteria; the conversion ratio of substrate catalyzed by the UDP-glucosyltransferase is up to 90%; and the produced rebaudioside M has a purity more than 990/% after being purified.
3. In the method for for preparing rebaudioside M by utilizing Saccharomyces cerevisiae in an enzymatic process, the produced rebaudioside M is quite save with a high purity and can be used directly as food additives without follow-up processing, thereby shortening the production cycle, increasing the yield, and decreasing the cost.

### BRIEF DESCRIPTION OF DRAWINGS

The invention is further described through FIGURES and detailed description.

FIG. 1 is a 1HNMR spectrogram of prepared rebaudioside M.

### DETAILED DESCRIPTION OF THE INVENTION

Hereinafter rebaudiosides A, D, and M are respectively termed as Reb A, Reb D, and Reb M for short.

The structural formulae of these three are described in formula I, II, and III respectively.

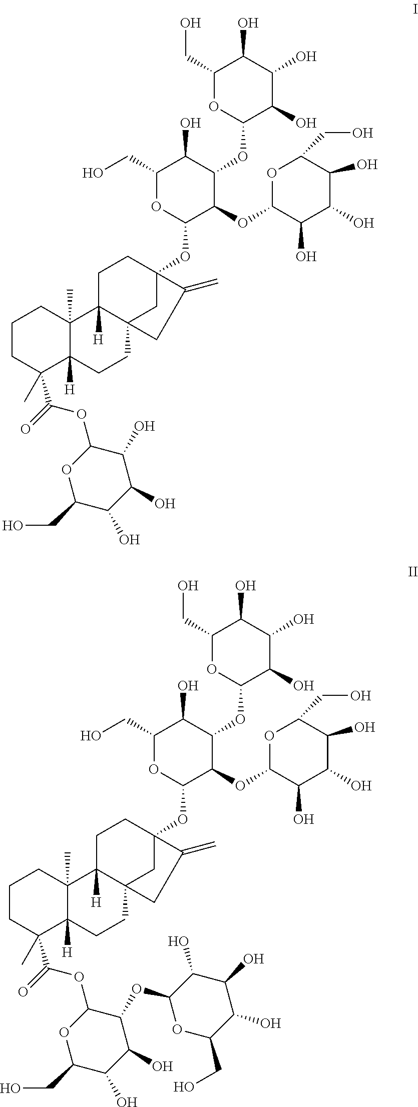

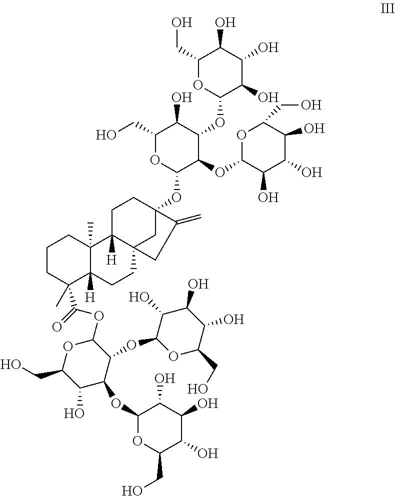

Four routes for synthesizing Reb M are provided by the present invention:

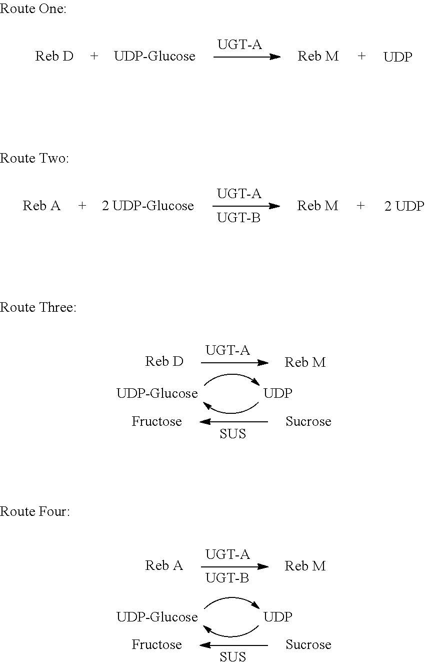

According to the present invention, the used -UGT-A or UGT-B may exist in the form of lyophilized enzyme powder or in the recombinant Saccharomyces cerevisiae.

The method for obtaining UGT-A or UGT-B is as follows:

A recombinant Saccharomyces cerevisiae expression strain of UGT-A or UGT-B is obtained by utilizing molecular cloning technique and genetic engineering technique; then the recombinant Saccharomyces cerevisiae is fermented to obtain recombinant cells containing UGT-A or UGT-B, or to obtain lyophilized powder of UGT-A or UGT-B.

The molecular cloning technique and genetic engineering technique described herein are known, unless otherwise specified. The molecular cloning technique may be found in ‘Molecular Cloning: A Laboratory Manual’ (3rd Edition) (J. Sambrook, 2005).

The expression steps of the recombinant strain herein constructed by employing genetic engineering technique are as follows:
(1) the gene fragment of UGT-A, UGT-B, or SUS is synthesized based on the amino acid sequence of UGT-A, UGT-B, or SUS, or the nucleotide sequence of gene UGT-A, UGT-B, or SUS; and then the gene fragment is ligated into vector pUC57;
(2) each gene fragment is inserted into enzyme cutting sites of the recombinant plasmids pEZADH2, pEZTEF1, and pEZADH1 through PCR, double digestion and ligation respectively so that each gene is placed under the control of different promoters;
(3) the expression vector is transformed into Saccharomyces cerevisiae BY4742 to obtain a recombinant Saccharomyces cerevisiae expression strain of UGT-A, UGT-B, or SUS.

Recombinant cells containing UGT-A or UGT-B, or lyophilized powder of UGT-A or UGT-B are prepared by utilizing the recombinant Saccharomyces cerevisiae expression strain containing UGT-A or UGT-B.

The specific steps of the foregoing method are described below via examples.

### Example 1 Preparing Recombinant Saccharomyces cerevisiae Cells Containing UGT-A

Based on the sequence of pYES2 plasmid, primers pYES2-AgeI-F(GATGATCCACTAGTAACCGGTAGAAGCCGCCG) and pYES2-AgeI-R(CGGCGGCTTCTACCGGTTACTAGTGGATCATC) were designed; an AgeI cutting site was introduced into the pYES2 plasmid through expand-loop PCR to obtain plasmid pYES2-AgeI.

INVSc2 genome was used as a template, primers ADH2-F(CACTAGTAACCGGTGCAAAACGTAGGGGC) and ADH2-R(GTCCAGCCCAAGCTTGTATTACGATATAG) were designed; after PCR amplification, the gene fragment of promoter ADH2 was obtained, then the gel was cut and recycled. The resulting gene fragment was digested with AgeI/HindIII; then the purified fragment was recovered; the fragment was ligated into corresponding enzyme cutting site of pYES2-AgeI by adding T4 ligase to obtain pEZTEF1 plasmid.

Based on the nucleotide sequence shown in SEQ ID NO. 2 in a sequence listing, the gene fragment of UGT-A was synthesized by gene and then was ligated into a vector pUC57 to obtain PUC57-UGT-A (Suzhou GENEWIZ Biotechnology Ltd.). Using pUC57-UGT-A as a template, gene UGT-A was obtained through PCR; HindIII and XbaI enzyme cutting sites were added to both ends of gene UGT-A; the UGT-A gene fragment was digested by restrictive endonuclease HindIII and XbaI; then the purified fragment was recovered; the fragment was ligated into respective enzyme cutting sites of pEZADH2 by adding T4 ligase to obtain pEZADH2-UGT-A plasmid; the plasmid was introduced into the Saccharomyces cerevisiae BY4742 to obtain a recombinant EZ-A strain.

The monoclonal plate was picked to SC-Ura+2% glucose medium, incubated at 30° C. at 200 rpm for 48 h, and EZ-A strain was inoculated to 50 ml YPD+1% glucose medium at 2% for oscillation at 30° C., and incubated at 200 rpm for 48 h. Cells were collected through centrifugation (4,000 rpm, 10 min); and the collected cells were resuspended with 5 ml of 0.1 mol/L phosphate buffer (pH 7.0) to obtain the recombinant cells containing UGT-A.

### Example 2 Preparing Lyophilized Powder of UGT-A

The recombinant cells containing UGT-A prepared in example 1 were ultrasonically disrupted in ice bath; the homogenate was centrifuged (8,000 rpm, 10 min); and the supernatant was collected and lyophilized for 24 h to obtain lyophilized powder of UGT-A.

### Example 3 Preparing Recombinant Saccharomyces cerevisiae Permeabilized Cells Containing UGT-A

Method 1: 1 g of wet cells of the recombinant cells of UGT-A prepared in Example 1 were weighed and resuspended to 20 ml of 75 mmol imidazole-0.1 mol/L KCl-10 mmol/L Mgcl2+1 ml terpene:ethanol (v/v)=1:4, 25° C., shaken at 160 rpm for 15 min; cells were collected through centrifugation (4,000 rpm, 10 min) and concentrated 10 times and resuspended in 0.1 mol/L Phosphoric acid buffer (pH 7.0) to obtain UGT-A recombinant Saccharomyces cerevisiae permeabilized cells for catalysis.

Method 2: The monoclonal plate was picked to an SC-Ura+2% glucose medium; then the medium was shaken for cultivating at 30° C. and 200 rpm for 48 h; EZ-A strain of 2% ratio was inoculated to 50 ml YPD+1% glucose medium; after the medium was shaken for cultivating at 30° C. and 200 rpm for 48 h, 500 μl of 200/% Triton X-100 (V/V) was added to the medium and the medium was shaken for another 2 h; cells were collected through centrifugation (4,000 rpm, 10 min); and the collected cells were resuspended with 5 ml of 0.1 mol/L phosphate buffer (pH 7.0) to obtain the recombinant Saccharomyces cerevisiae permeabilized cells containing UGT-A for catalysis.

### Example 4 Preparing Recombinant Saccharomyces cerevisiae Cells Containing UGT-B

Based on the sequence of pYES2 plasmid, primers pYES2-AgeI-F(GATGATCCACTAGTAACCGGTAGAAGCCGCCG) and pYES2-AgeI-R(CGGCGGCTTCTACCGGTTACTAGTGGATCATC) were designed; an AgeI cutting site was introduced into the pYES2 plasmid through expand-loop PCR to obtain plasmid pYES2-AgeI.

INVSc2 genome was used as a template, primers TEF1-F(CCACTAGTAACCGGTCACACACCATAGCTTC) and TEF1-R(GTCCAGCCCAAGCTTTGTAATTAAAACTTAG) were designed; after PCR amplification, the gene fragment of promoter TEF1 was obtained, then the gel was cut and recycled. The resulting gene fragment was digested with AgeI/HindIII; then the purified fragment was recovered; the fragment was ligated into corresponding enzyme cutting site of pYES2-AgeI by adding T4 ligase to obtain pEZTEF1 plasmid.

Based on the nucleotide sequence shown by SEQ ID NO. 4 in a sequence listing, the gene fragment of UGT-B was synthesized by gene and then was ligated into a pUC57 vector to obtain PUC57-UGT-B (Suzhou GENEWIZ Biotechnology Ltd.). Using pUC57-UGT-B as a template, gene UGT-B was obtained through PCR; HindIII and XbaI enzyme cutting sites were added to both ends of gene UGT-B; the gene UGT-B fragment was digested by restrictive endonuclease HindIII and XbaI; then the purified fragment was recovered; the fragment was ligated into respective enzyme cutting site of pEZTEF1 by adding T4 ligase to obtain pEZTEF1-UGT-B plasmid; the plasmid was introduced into the Saccharomyces cerevisiae BY4742 to obtain a recombinant EZ-B strain.

The monoclonal plate was picked to SC-Ura+2% glucose medium, incubated at 30° C. at 200 rpm for 48 h, and EZ-A strain was inoculated to 50 ml YPD+1% glucose medium at 2% for oscillation at 30° C., and incubated at 200 rpm for 48 h. Cells were collected through centrifugation (4,000 rpm, 10 min); and the collected cells were resuspended with 5 ml of 0.1 mol/L phosphate buffer (pH 7.0) to obtain the recombinant cells containing UGT-B.

### Example 5 Preparing Lyophilized Powder of UGT-B

The recombinant cells containing UGT-B prepared in example 4 were ultrasonically disrupted in ice bath; the homogenate was centrifuged (8,000 rpm, 10 min); and the supernatant was collected and lyophilized for 24 h to obtain lyophilized powder of UGT-B.

### Example 6 Preparing Recombinant Saccharomyces cerevisiae Permeabilized Cells Containing UGT-B

Method 1: 1 g of wet cells of the recombinant cells of UGT-B prepared in Example 1 were weighed and resuspended to 20 ml of 75 mmol imidazole-0.1 mol/L KCl-10 mmol/L Mgcl2+1 ml terpene:ethanol (v/v)=1:4, 25° C., shaken at 160 rpm for 15 min; cells were collected through centrifugation (4,000 rpm, 10 min) and concentrated 10 times and resuspended in 0.1 mol/L Phosphoric acid buffer (pH 7.0) to obtain UGT-B recombinant Saccharomyces cerevisiae permeabilized cells for catalysis.

Method 2: The monoclonal plate was picked to an SC-Ura+2% glucose medium; then the medium was shaken for cultivating at 30° C. and 200 rpm for 48 h; EZ-B strain of 2% ratio was inoculated to 5 ml YPD+1% glucose medium; after the medium was shaken for cultivating at 30° C. and 200 rpm for 48 h, 500 μl of 20% Triton X-100 (V/V) was added to the medium and the medium was shaken for another 2 h; cells were collected through centrifugation (4,000 rpm, 10 min); and the collected cells were resuspended with 5 ml of 0.1 mol/L phosphate buffer (pH 7.0) to obtain the recombinant Saccharomyces cerevisiae permeabilized cells containing UGT-B for catalysis.

### Example 7 Preparing Recombinant Saccharomyces cerevisiae Cells Containing USU

Based on the sequence of pYES2 plasmid, primers pYES2-AgeI-F(GATGATCCACTAGTAACCGGTAGAAGCCGCCG) and pYES2-AgeI-R(CGGCGGCTTCTACCGGTTACTAGTGGATCATC) were designed; an AgeI cutting site was introduced into the pYES2 plasmid through expand-loop PCR to obtain plasmid pYES2-AgeI.

INVSc2 genome was used as a template, primers ADH1-F(TCCACTAGTAACCGGTCTCCCTAACATGTAGG) and ADH1-R(GTCCAGcccAAGCTTAGTTGATTGTATGC) were designed; after PCR amplification, the gene fragment of promoter ADH1 was obtained, then the gel was cut and recycled. The resulting gene fragment was digested with AgeI/HindIII; then the purified fragment was recovered; the fragment was ligated into corresponding enzyme cutting site of pYES2-AgeI by adding T4 ligase to obtain pEZADH1 plasmid.

Based on the nucleotide sequence shown in SEQ ID NO. 2 or the nucleotide sequence shown by SEQ ID NO. 6 in a sequence listing, the gene fragment of SUS was synthesized by gene and then was ligated into a pUC57 vector to obtain PUC57-SUS (Suzhou GENEWIZ Biotechnology Ltd.). Using pUC57-SUS as a template, gene SUS was obtained through PCR; HindIII and XbaI enzyme cutting sites were added to both ends of gene SUS; the gene SUS fragment was digested by restrictive endonuclease HindIII and XbaI; then the purified fragment was recovered; the fragment was ligated into respective enzyme cutting site of pEZADH1 by adding T4 ligase to obtain pEZADH1-SUS plasmid; the plasmid was introduced into the Saccharomyces cerevisiae BY4742 to obtain a recombinant EZ-S strain.

The monoclonal plate was picked to SC-Ura+2% glucose medium, incubated at 30° C. at 200 rpm for 48 h, and EZ-S strain was inoculated to 50 ml YPD+1% glucose medium at 2% for oscillation at 30° C., and incubated at 200 rpm for 48 h. Cells were collected through centrifugation (4,000 rpm, 10 min), and the collected cells were resuspended with 5 ml of 0.1 mol/L phosphate buffer (pH 7.0) to obtain the recombinant cells containing SUS for catalysis.

### Example 8 Preparing Lyophilized Powder of SUS

The recombinant cells containing SUS prepared in example 7 were ultrasonically disrupted in ice bath; the homogenate was centrifuged (8,000 rpm, 10 min); and the supernatant was collected and lyophilized for 24 h to obtain lyophilized powder of SUS.

### Example 9 Preparing Recombinant Saccharomyces cerevisiae Permeabilized Cells Containing SUS

Method 1: 1 g of wet cells of the recombinant cells of SUS prepared in Example 7 were weighed and resuspended to 20 ml of 75 mmol imidazole-0.1 mol/L KCl-10 mmol/L Mgcl2+1 ml terpene:ethanol (v/v)=1:4, 25° C., shaken at 160 rpm for 15 min; cells were collected through centrifugation (4,000 rpm, 10 min) and concentrated 10 times and resuspended in 0.1 mol/L Phosphoric acid buffer (pH 7.0) to obtain SUS recombinant Saccharomyces cerevisiae permeabilized cells for catalysis.

Method 2: The monoclonal plate was picked to an SC-Ura+2% glucose medium; then the medium was shaken for cultivating at 30° C. and 200 rpm for 48 h; EZ-S strain of 2% ratio was inoculated to 50 ml YPD+1% glucose medium; after the medium was shaken for cultivating at 30° C. and 200 rpm for 48 h, 500 μl of 200/% Triton X-100 (V/V) was added to the medium and the medium was shaken for another 2 h; cells were collected through centrifugation (4.000 rpm, 10 min); and the collected cells were resuspended with 5 ml of 0.1 mol/L phosphate buffer (pH 7.0) to obtain the recombinant Saccharomyces cerevisiae permeabilized cells containing SUS for catalysis.

### Example 10 Synthesizing Reb M Using Reb D as a Substrate in an Enzymatic Process

100 mL of 0.1 mol/L phosphate buffer (pH 7.0), 0.224 g of UDP, 34.2 g of sucrose, 0.2 g of Reb D, 1 g lyophilized powder of UGT-A and 0.4 g lyophilized powder of SUS were sequentially added to the reaction system and uniformly mixed.

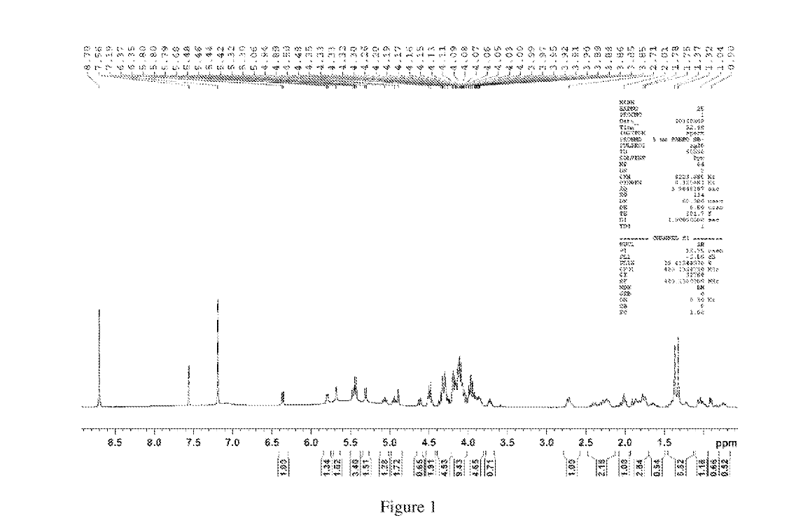

The mixture was placed in a 37° C. water bath and stirred at 200 rpm for 18 h. After the reaction was complete, 300 μl of the reaction liquid was taken and added therein 600 μl of 3% formic acid and mixed; centrifuged at 10,000 rpm for 5 min; and high performance liquid chromatography was used to detect the supernatant filtration membrane (chromatographic conditions: column:Agilent eclipse sb-C18 4.6×150 mm; detection wavelength: 210 nm; mobile phase: methanol:water=68%:32%; flow rate: 1.0 mL/min; column temperature: 30° C.). The conversion ratio of Reb D was more than 900/o. After the supernatant was purified through post-processing such as isolating with resin and crystallizing, 0.1 g of Reb M was obtained with a purity thereof greater than 99%. The 1HNMR spectrum thereof is shown in FIG. 1.

### Example 11 Synthesizing Reb M Using Reb a as a Substrate in an Enzymatic Process

100 mL of o.1 mol/L phosphate buffer (pH 7.0), 0.18 g of UDP, 41.04 g of sucrose, 0.2 g of Reb A, 2 g lyophilized powder of UGT-A, 1 g lyophilized powder of UGT-B, and 0.5 g lyophilized powder of SUS were sequentially added to the reaction system and uniformly mixed. The mixture was placed in a 37° C. water bath and stirred at 200 rpm for 18 h. After the reaction was complete, 300 μl of the reaction liquid was taken and added therein 600 μl of 3% formic acid and mixed; centrifuged at 10,000 rpm for 5 min; and high performance liquid chromatography was used to detect the supernatant filtration membrane (chromatographic conditions: column:Agilent eclipse sb-C18 4.6×150 mm; detection wavelength: 210 nm; mobile phase: methanol:water=68%:32%; flow rate: 1.0 mL/min; column temperature: 30° C.). The conversion ratio of Reb A was more than 95%. After the supernatant was purified by post-processing such as isolating by resin and crystallizing, 0.09 g of Reb M was obtained, and the purity of which was greater than 99%.

### Example 12 Whole Cell Synthesizing Reb M Using Reb D as a Substrate

100 mL of 0.1 mol/L phosphate buffer (pH 7.0), 0.54 g of UDP-G, 0.1 g of Reb D, and 1 g whole cells of UGT-A were sequentially added to the reaction system and uniformly mixed. The mixture was placed in a 37° C. water bath and stirred at 200 rpm for 18 h. After the reaction was complete, 300 μl of the reaction liquid was taken and added therein 6001 of 3% formic acid and mixed; centrifuged at 10,000 rpm for 5 min; and high performance liquid chromatography was used to detect the supernatant filtration membrane (chromatographic conditions: column:Agilent eclipse sb-C18 4.6×150 mm; detection wavelength: 210 nm; mobile phase: methanol:water=68%:32%; flow rate: 1.0 mL/min; column temperature: 30° C.). The conversion ratio of Reb D was more than 90%. After the supernatant was purified by post-processing such as isolating by resin and crystallizing, 0.09 g Reb M was obtained, and the purity of which was higher than 99%.

### Example 13 Whole Cell Synthesizing Reb M Using Reb a as a Substrate

100 mL of 0.1 mol/L phosphate buffer (pH 7.0), 1.08 g of UDP-G, 0.1 g of Reb A, 2 g whole cells of UGT-A, and 1 g whole cells of UGT-B were sequentially added to the reaction system and uniformly mixed. The mixture was placed in a 37° C. water bath and stirred at 200 rpm for 18 h. After the reaction was complete, 300 μl of the reaction liquid was taken and added therein 600 μl of 3% formic acid and mixed; centrifuged at 10,000 rpm for 5 min; and high performance liquid chromatography was used to detect the supernatant filtration membrane (chromatographic conditions: column:Agilent eclipse sb-C18 4.6×150 mm; detection wavelength: 210 nm; mobile phase: methanol:water=68%:32%; flow rate: 1.0 mL/min; column temperature: 30° C.). The conversion ratio of Reb A was more than 40%. After the supernatant was purified by post-processing such as isolating by resin and crystallizing, 0.04 g of Reb M was obtained, and the purity of which was greater than 99%.

### Example 14 Method for Isolating and Purifying Reb M

1.5 L of deionized water was added to 500 ml of reaction solution, then the solution was heated at 55° C. for 1 hour, sonicated, and centrifuged at 6,700 rpm for 30 min. The obtained supernatant was sample A. After centrifugation and precipitation, 500 ml of water was added; heated at 55° C. for 0.5 hour and sonicated, centrifuged at 6700 rpm for 30 minutes, and the supernatant was sample B. The sample A was mixed with the sample B to obtain sample C, which was isolated with macroporous adsorption resin (AB-8). Water was used to flush a 4-column volume and then 70% ethanol was used to elute a 3.5-column volume to obtain crude Reb M solution. The foregoing crude solution was distilled under reduced pressure (40-50° C.) to about 50 mL of a remaining volume; the remaining solution was centrifuged at 9900 rpm for 10 min; the supernatant was discarded. The precipitation was washed with 20 mL of water, then the obtained solution was centrifuged at 9900 rpm for 10 min; the supernatant was discarded. The obtained precipitation was suspended with 50%° ethanol aqueous solution; the suspension was heated to 65° C. for dissolving; then an equal volume of water was added to the obtained solution until ethanol concentration was 25%. The solution was gradually cooled to room temperature; after a solid was precipitated, the solution was filtered by suction and dried in vacuo to obtain a Reb M sample with a purity more than 99%.

### Example 15

The substrate conversion ratios in the processes of catalytically preparing Reb M by the UDP-glucosyltransferase gene in different plasmids and host bacteria were as shown in Table 1-2, wherein the glucosyl donors are UDP-G.

Table 1 shows the substrate conversion rates in the process of catalytically preparing Reb M by UDP-glucosyltransferase gene under the action of pYES2, Gal promoter and different host bacteria.

| Host | Plasmid | Catalytic reaction | Conversion ratio |
|---|---|---|---|
| CEN.PK2-1C | pYES2-UGTA | Reb D--Reb M | 14% |
| BY4742 | Gal promoter | | 11% |
| BJ5456 | needing induction | | 0 |
| CEN.PK2-1C | pYES2-UGTB | Reb A--Reb D | 4% |
| BY4742 | Gal promoter | | 1% |
| BJ5456 | needing induction | | 0 |

Table 2 shows the substrate conversion ratios in the processes for catalytically preparing Reb M in CEN.PK2-1C or BY4742 by UDP-glucosyltransferase gene via the pYES2 plasmid containing different promoters.

| | | | Conversion |
|---|---|---|---|
| Host | Plasmid | Catalytic reaction | ratio |
| CEN.PK2-1C | pEZADH1-UGTA | Reb D--Reb M | 14% |
| BY4742 | ADH1 promoter | | 13% |
| CEN.PK2-1C | pEZADH2-UGTA | Reb D--Reb M | 81% |
| BY4742 | ADH2 promoter | | 95% |
| CEN.PK2-1C | pEZTEF1-UGTA | Reb D--Reb M | 55% |
| BY4742 | TEF1 promoter | | 45% |
| CEN.PK2-1C | pEZGPD-UGTA | Reb D--Reb M | 56% |
| BY4742 | GPD promoter | | 66% |
| CEN.PK2-1C | pEZADH1-UGTA | Reb A--Reb D | 11% |
| BY4742 | ADH1 promoter | | 11% |
| CEN.PK2-1C | pEZADH2-UGTA | Reb A--Reb D | 12% |
| BY4742 | ADH2 promoter | | 34% |
| CEN.PK2-1C | pEZTEF1-UGTA | Reb A--Reb D | 5% |
| BY4742 | TEF1 promoter | | 52% |
| CEN.PK2-1C | pEZGPD-UGTA | Reb A--Reb D | 38% |
| BY4742 | GPD promoter | | 38% |

Obviously, the foregoing examples are merely for clearly describing the illustrated examples, and not intended to limit embodiments. Other variations or modifications of the various forms may be made on the basis of the above description for those of ordinary skill in the art. It is not required or possible to exhaust all embodiments. And obvious variations or alterations derived therefrom still fall within the protection scope of the present invention patent.

1-13. (canceled)

14. A method for preparing rebaudioside M, the method comprising reacting rebaudioside D in a reaction solution with a glucosyl donor in the presence of a first UDP-glucosyltransferase or a first recombinant Saccharomyces cerevisiae comprising the first UDP-glucosyltransferase,
wherein the first UDP-glucosyltransferase has an amino acid sequence according to SEQ ID NO: 1; and
wherein the first UDP-glucosyltransferase is expressed from a first pYES2 expression vector comprising a first strong promoter in the first recombinant Saccharomyces cerevisiae.

15. The method of claim 14, wherein the rebaudioside D is prepared by reacting rebaudioside A in the reaction solution with the glucosyl donor in the presence of a second UDP-glucosyltransferase or a second recombinant Saccharomyces cerevisiae comprising the second UDP-glucosyltransferase,
wherein the second UDP-glucosyltransferase has an amino acid sequence according to SEQ ID NO: 3; and
wherein the second UDP-glucosyltransferase is expressed from a second pYES2 expression vector comprising a second strong promoter in the second recombinant Saccharomyces cerevisiae.

16. The method of claim 14, wherein the first recombinant Saccharomyces cerevisiae is Saccharomyces cerevisiae BY4742.

17. The method of claim 15, wherein the second recombinant Saccharomyces cerevisiae is Saccharomyces cerevisiae BY4742.

18. The method of claim 14, wherein the first strong promoter and the second strong promoter are each independently ADH2 or TEF1.

19. The method of claim 15, wherein the first strong promoter and the second strong promoter are each independently ADH2 or TEF1.

20. The method of claim 14, wherein the first UDP-glucosyltransferase is provided as a lyophilized powder comprising the first UDP-glucosyltransferase.

21. The method of claim 15, wherein the second UDP-glucosyltransferase is provided as a lyophilized powder comprising the second UDP-glucosyltransferase.

22. The method of claim 14, wherein the rebaudioside M is prepared by reacting rebaudioside D with the glucosyl donor in the presence of the first recombinant Saccharomyces cerevisiae comprising the first UDP-glucosyltransferase, wherein the first recombinant Saccharomyces cerevisiae has been treated with a cell-permeating agent prior to reacting the rebaudioside D with the glucosyl donor.

23. The method of claim 15, wherein the rebaudioside D is prepared by reacting rebaudioside A with the glucosyl donor in the presence of the second recombinant Saccharomyces cerevisiae comprising the second UDP-glucosyltransferase, wherein the second recombinant Saccharomyces cerevisiae has been treated with a cell-permeating agent prior to reacting the rebaudioside A with the glucosyl donor.

24. The method of claim 14, wherein the glucosyl donor is UDP-glucose, or a UDP-glucose regeneration system comprising sucrose, sucrose synthetase, and UDP.

25. The method of claim 15, wherein the glucosyl donor is UDP-glucose, or a UDP-glucose regeneration system comprising sucrose, sucrose synthetase, and UDP.

26. The method of claim 24, wherein the sucrose synthetase is provided as a lyophilized powder comprising the sucrose synthetase, and wherein the sucrose synthetase was expressed in a third recombinant Saccharomyces cerevisiae prior to preparing the lyophilized powder.

27. The method of claim 25, wherein the sucrose synthetase is provided as a lyophilized powder comprising the sucrose synthetase, and wherein the sucrose synthetase was expressed in a third recombinant Saccharomyces cerevisiae prior to preparing the lyophilized powder.

28. The method of claim 24, wherein the sucrose synthetase is provided as a third recombinant Saccharomyces cerevisiae comprising the sucrose synthetase; and
wherein the third recombinant Saccharomyces cerevisiae is treated with a cell-permeating agent.

29. The method of claim 25, wherein the sucrose synthetase is provided as a third recombinant Saccharomyces cerevisiae comprising the sucrose synthetase; and
wherein the third recombinant Saccharomyces cerevisiae is treated with a cell-permeating agent.

30. The method of claim 14, wherein reacting rebaudioside D with the glucosyl donor in the presence of the first UDP-glucosyltransferase or the first recombinant Saccharomyces cerevisiae comprising the first UDP-glucosyltransferase is carried out in an aqueous system at a temperature of 4° C. to 50° C. and a pH of 5.0 to 9.0 for 18 hours.

31. The method of claim 30, wherein the aqueous system comprises phosphate buffer at a temperature of 37° C. and a pH of 7.0.

32. The method of claim 15, wherein reacting rebaudioside A with the glucosyl donor in the presence of the second UDP-glucosyltransferase or the second recombinant Saccharomyces cerevisiae comprising the second UDP-glucosyltransferase is carried out in an aqueous system at a temperature of 4° C. to 50° C. and a pH of 5.0 to 9.0 for 18 hours.

33. The method of claim 32, wherein the aqueous system comprises phosphate buffer at a temperature of 37° C. and a pH of 7.0.
